(19) United States
(12) Patent Application Publication
(10) Pub. No.: US 2006/0247716 A1
(43) Pub. Date: Nov. 2, 2006

(54) Radially sealing set screw

(75) Inventors: Benjamin Fruland, Plymouth, MN (US); Nick A. Youker, River Falls, WI (US); William J. Linder, Golden Valley, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/117,961

(22) Filed: Apr. 29, 2005

(51) Int. Cl. A61N 1/375

(52) U.S. Cl. 607/36

(57) ABSTRACT

This document discusses, among other things, a medical device including an implantable medical device casing including an opening having an inner surface, a screw, and a non-conductive sleeve configured to fit within the opening, the non-conductive sleeve having an outer surface and an inner surface. An example screw includes a top portion including a driver interface, and a threaded bottom portion, at least one of the top portion and the bottom portion being non-conductive. The sleeve and implantable medical device casing are adapted to sealingly engage with the screw when assembled. In another example, a screw includes a compressible rib that forms a seal with a medical device. In an example method, a screw is pressed against a compressible component such as a sleeve or rib to form a seal against a medical device.

### TECHNICAL FIELD

This patent document pertains generally to sealed connections for medical devices and more particularly, but not by way of limitation, to radially sealing set screws for electrical insulation from body fluids.

### BACKGROUND

Implantable medical devices such as pacers and defibrillators commonly include one or more screws. For example, leads are typically coupled to a pacer or defibrillator using one or more set screws.

Penetration of body fluids around a lead connection can lead to corrosion or other problems. It is also desirable to avoid current leakage through electrical pathways created by body fluids. In some instances, set screws are accessible from the outside of a medical device. This allows leads to be connected or disconnected to a device.

In one prior art seal configuration, a screw is assembled into a device under a seal plug that includes a slit, which allows for passage of a wrench through the plug to tighten the set screw. The plug is configured so that the slit closes after the wrench is removed, thereby sealing the set screw below the plug from body fluids above the plug. An example of such a seal plug is described in U.S. Pat. No. 4,479,489. Another example of a seal is described in U.S. Pat. No. 3,908,668.

### BRIEF DESCRIPTION OF THE DRAWINGS

In the drawings, which are not necessarily drawn to scale, like numerals describe substantially similar components throughout the several views. The drawings illustrate generally, by way of example, but not by way of limitation, various embodiments discussed in the present document.

FIG. 1 is a cross-sectional view of a medical device including a screw and a sleeve.

FIG. 2 is an enlarged view of the medical device of FIG. 2.

FIG. 3 is a side view of a screw that has ribs extending around an outer surface.

FIG. 4 is a perspective view of the screw of FIG. 3.

FIG. 5 is a top view of the screw of FIG. 3.

FIG. 6 is a cross-sectional view the screw of FIG. 3.

FIG. 7 is a partially cut-away perspective view of the screw of FIG. 3.

FIG. 8 is a partial cross-sectional view of an alternative screw inserted in an opening in a medical device.

FIG. 9 is a perspective view of another example screw.

FIG. 10 is a cross-sectional view of a screw, a medical device casing, and a conductor.

FIG. 11 is a cross-sectional view of a screw configured in a sleeve that has an inwardly-extending rib configured to seal against the screw.

FIG. 12 is a cross-sectional view of a screw configured in a sleeve.

FIG. 13 is a cross-sectional view of a device and another example screw having an external drive interface.

FIG. 14 is a cross-sectional view of a device, a sleeve, and another example screw having an external drive interface.

FIG. 15 is a flow chart that illustrates an example method.

### DETAILED DESCRIPTION

The following detailed description includes references to the accompanying drawings, which form a part of the detailed description. The drawings show, by way of illustration, specific embodiments in which the invention may be practiced. These embodiments are also referred to herein as “examples.”

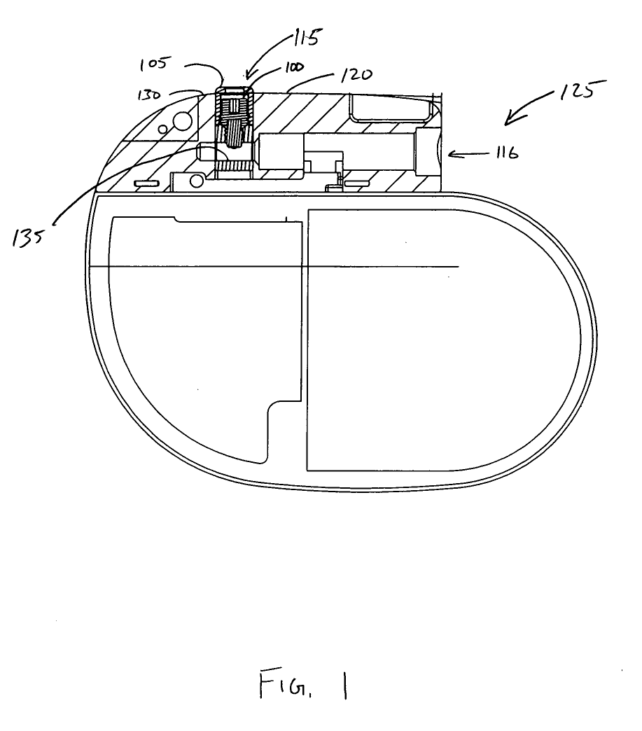

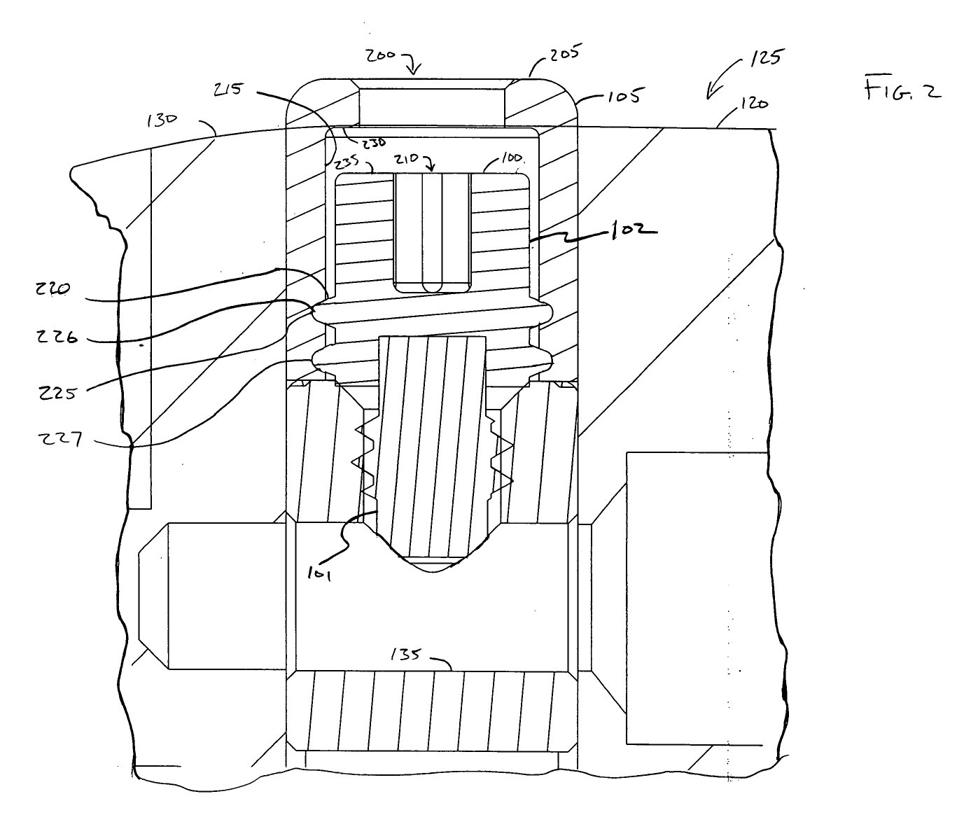

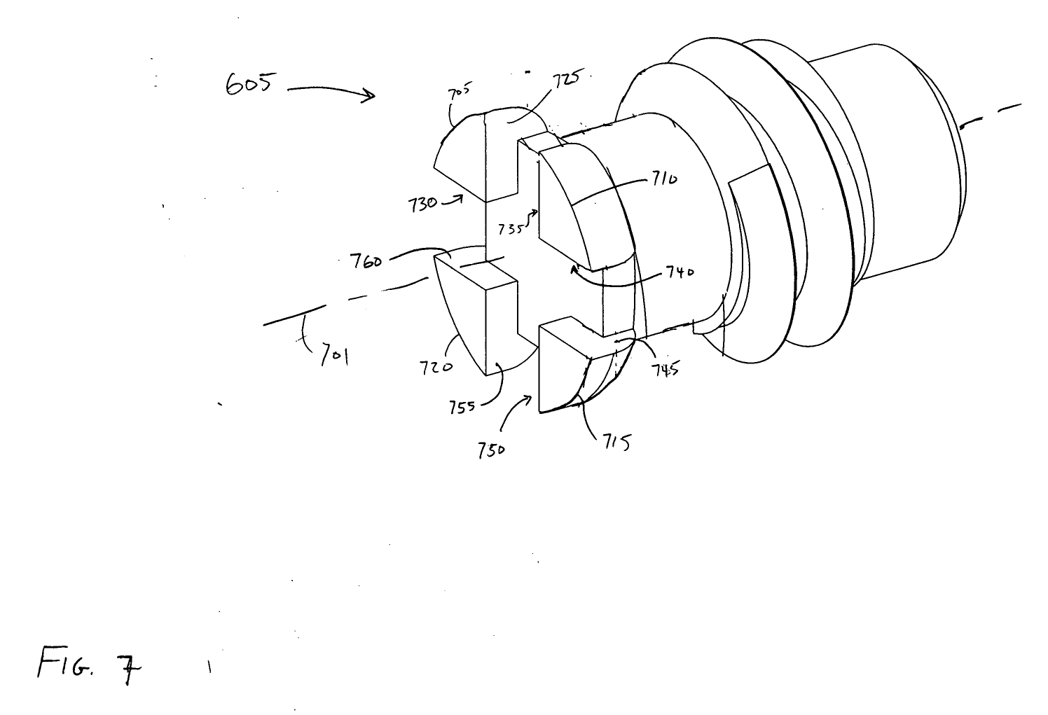

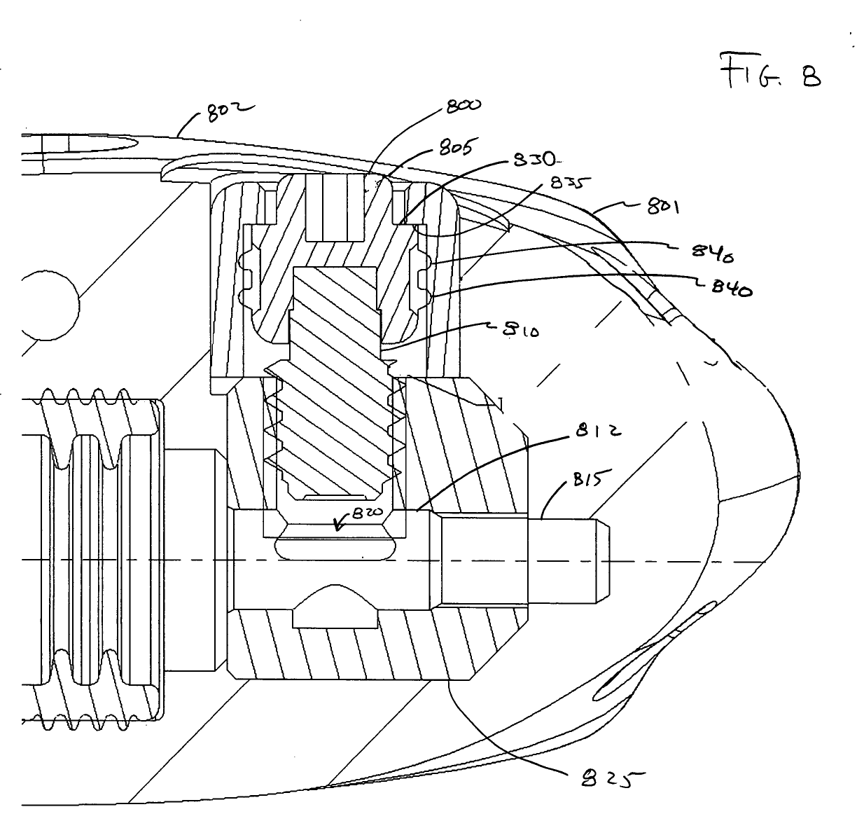

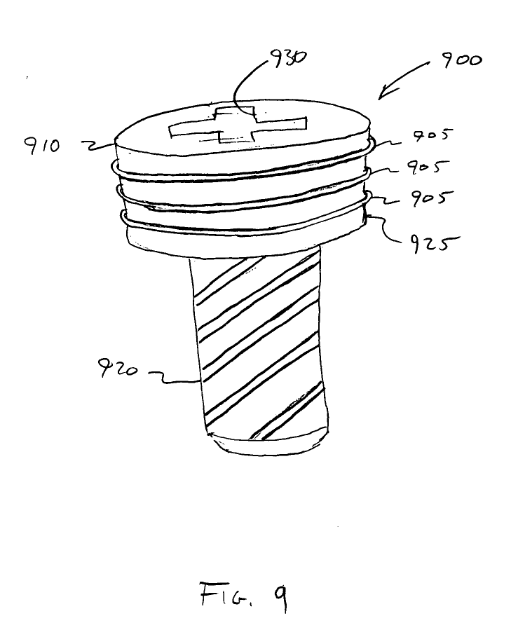

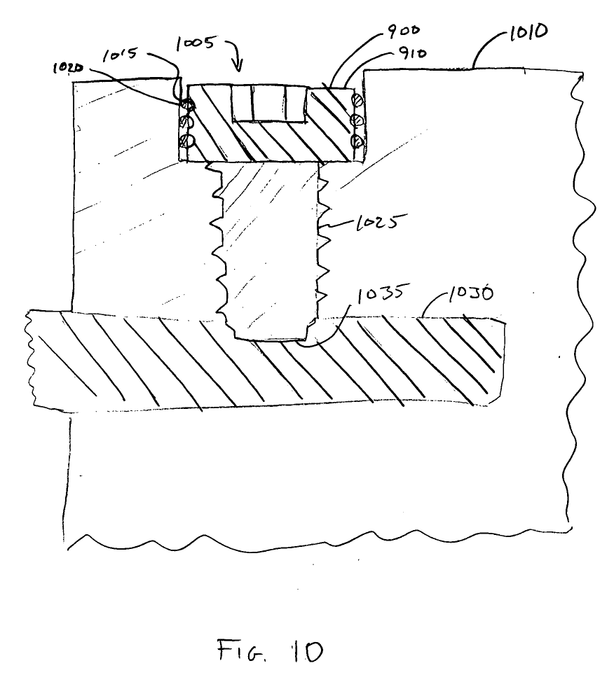

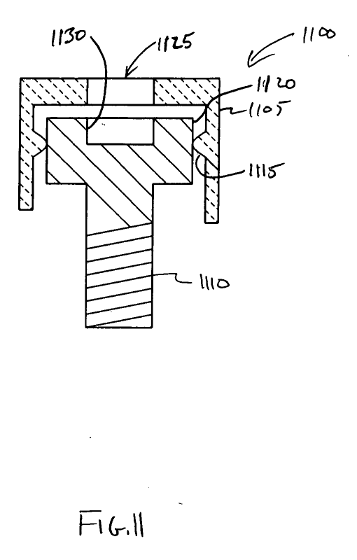

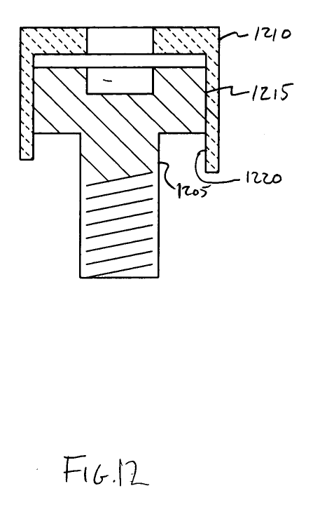

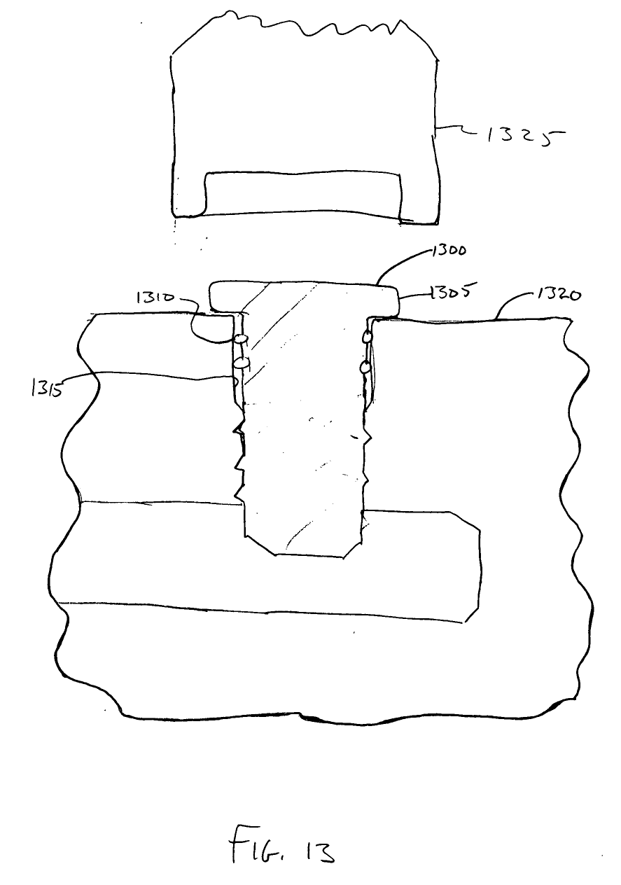

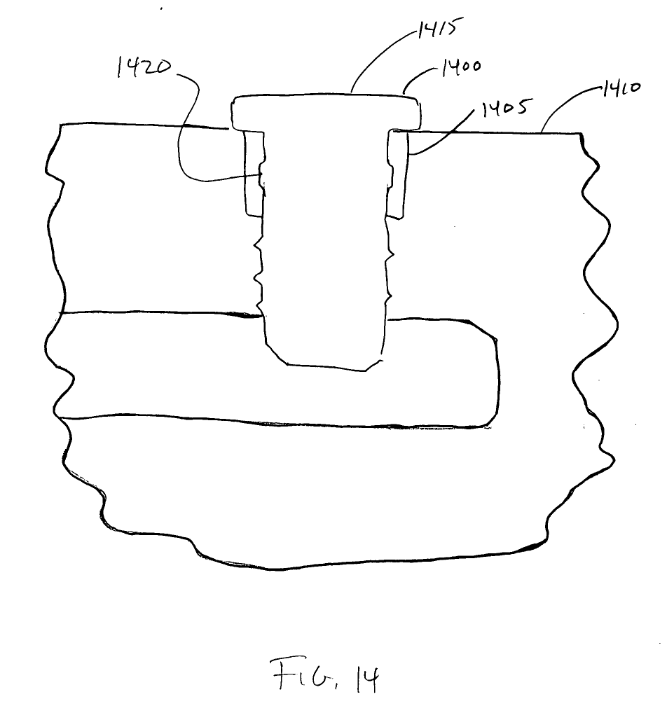

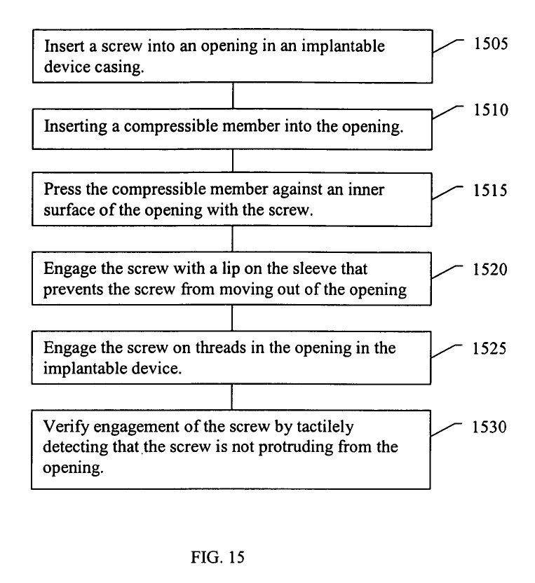

An example screw includes a non-conductive portion, such as a non-conductive head or a non-conductive shaft. In an example, the screw is a set screw that coupled a conductor to an object, such as a medical device casing. In an example shown in FIGS. 1-6, the screw includes one or more protruding features, such as a rib, that presses against a non-conductive sleeve in an opening in a medical device. In an example, an insulative head is insert-molded over a shaft. An example screw that can be formed by insert-molding is shown in cross-section in FIG. 2. FIG. 7 shows a shaft that includes a structure that facilitates torque transfer between a molded head and the shaft. FIG. 8 shows another example screw that can be formed by insert-molding a non-conductive head over a shaft. FIGS. 9 and 10 show a screw having a compressible rib that is sealable against an opening in a medical device casing, for example. FIG. 11 shows another example configuration in which a sleeve has an inwardly-extending rib. FIG. 12 shows another example in which a screw fits with a press-fit or interference fit in a sleeve. FIGS. 13 and 14 show example screws that have external drives that interface with a socket wrench. FIG. 15 is a flow chart that illustrates a method.

Referring now to FIGS. 1 and 2, a screw 100 and sleeve 105 are insertable into an opening 115 in a casing 120 for a medical device 125. In an example, the medical device 125 is an implantable device such as a pacer or defibrillator. In an example, the screw 100 is a set screw that secures a conductor in a header portion 130 of the casing 120. Advancing the screw on threads in the casing 120 causes the screw to engage a conductor, such as a lead connector inserted through a second opening 116 in the casing. A shaft portion 101 of the screw 100 presses the conductor against an electrical contact 135 in the device 125 and fixes the conductor in the device. In an example, the shaft 101 extends through the electrical contact. In another example, the medical device includes a second electrical contact that touches the shaft. While the head portion 102 of the screw is shown having a larger cross-section than the shaft, in alternative examples the head has the same cross-section (e.g. same diameter) as the shaft, or is smaller than the shaft. In an example, the shaft is conductive. In implantable devices, it is desirable to avoid electrical conduction through the opening 115 in the casing 120, for example through the screw 100 or through fluid in or around the opening. It is also desirable to avoid fluid contact with the electrical contact 135 or the conductor to avoid corrosion or other problems.

Referring now to FIG. 2, in an example, the sleeve 105 includes an opening 200 in a top surface 205, through which a wrench can be inserted and engaged with a driver interface 210 on the screw 100. The screw 100 presses against an inner surface 215 of the sleeve 105 and sealably engages the sleeve. In an example, the screw 100 includes an outer surface 220 that has at least one wide portion 225 that has a larger outer dimension (e.g. diameter) than the inner surface 215 of the sleeve. In an example, the screw includes a raised rib 226 that includes the wide portion 225. In the example shown in FIG. 2, the screw includes two raised ribs 226, 227.

In an example, the inner surface 215 of the sleeve is cylindrical. In an example, both raised ribs 226, 227 have the same outer dimension, e.g. the same outer diameter. In another example, one or more ribs has an outer dimension that is larger than the outer dimension of at least one other rib. The raised rib 226 (or other wide portion of the screw) presses against the sleeve 105 and sealably engages the sleeve.

In an example, the sleeve 105 is compressible, and a rib 226 locally compresses the sleeve 105 to form a seal between the rib and the sleeve. In an example, the sleeve 105 includes silicone. The rib 226 locally increases the pressure on the sleeve 105 while still allowing the screw 100 to move past the sleeve as the screw advances on the threads. In an example, the screw includes only one rib. In other example, the screw includes multiple ribs, such as 2, 3, or 4 ribs, for example. In an alternative example, a rib is thicker (along the screw axis) or thinner than the rib shown in FIGS. 1-2. In another example, the screw rib is taller (radially relative to the outer surface of the screw) or shorter than the rib shown in the figures. The amount of interference with the sleeve can be controlled by varying the thickness of the ribs and the diameter (or height) of the ribs. Sealing and assembly characteristics can be controlled by varying the amount of interference between the screw and the sleeve.

In an example, the sleeve includes a lip 230 that extends inwardly over the top surface 235 of the screw 100. The lip 230 prevents the screw from falling out of the device 125 if the screw is disengaged with the threads. In an example, the lip defines the opening 200 in the sleeve 105. In an example, the opening is circular and has a diameter that is smaller than an outer diameter of the top surface 235 of the screw 100. In another example, the opening is slit shaped or oblong.

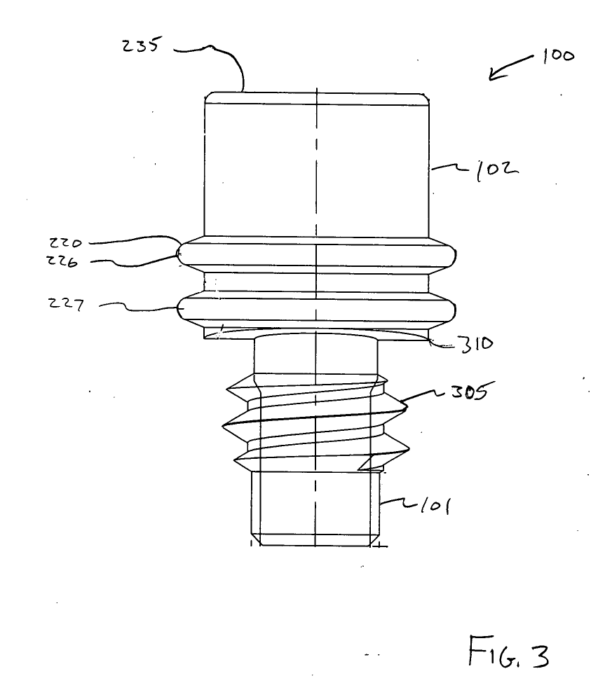

Referring now to FIG. 3, a front view of the example screw 100 is shown.

The shaft 101 includes external threads 305 which are engageable on internal threads in the medical device. In an example, the head 102 includes ribs 226, 227 near a bottom end 310 of the head. In an example, placing the ribs low on the head allows the ribs to seal throughout its range of travel. In other examples, the ribs are located elsewhere on the screw, including for example a middle portion or upper portion of the screw. In an example, the shaft 101 is conductive to allow electrical conduction through the screw between an electrical connector and an electrical contact 135 in the medical device shown in FIG. 1.

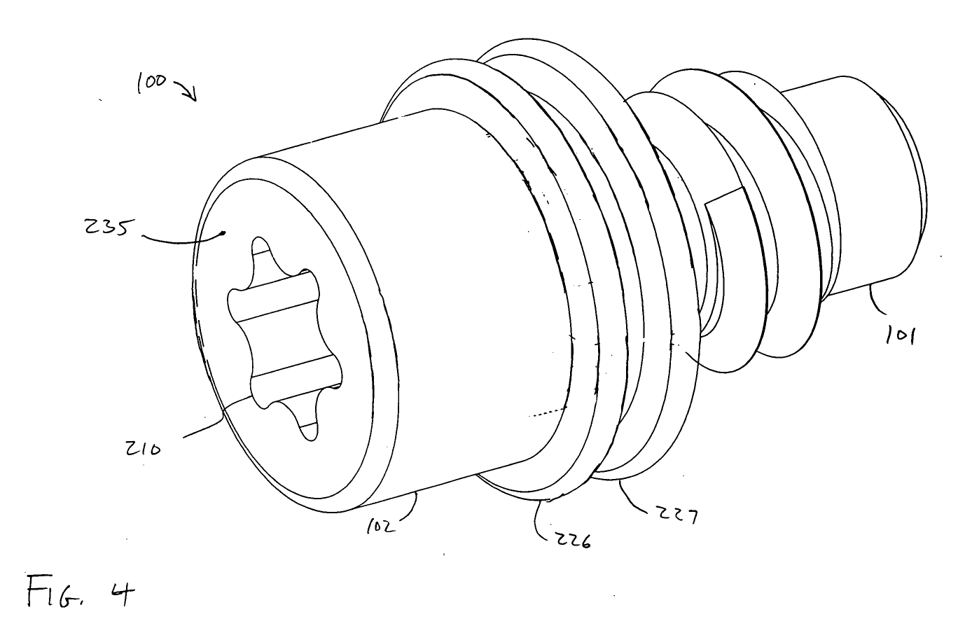

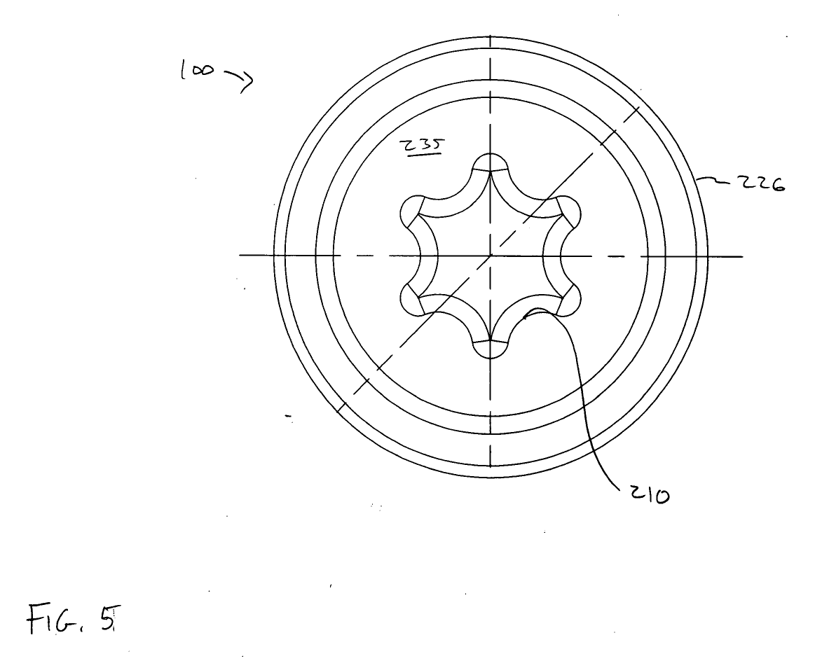

Referring now to FIGS. 4 and 5, a perspective view of the screw is shown in FIG. 4, and a top view of the screw is shown in FIG. 5. FIGS. 4 and 5 show the drive interface, which is an internal drive. In an example, the drive is a six lobed drive. In an alternative example, the drive is cross-shaped, as shown in FIG. 9. In another example, the drive is an external drive that interfaces with a socket wrench, as shown in FIGS. 13 and 14.

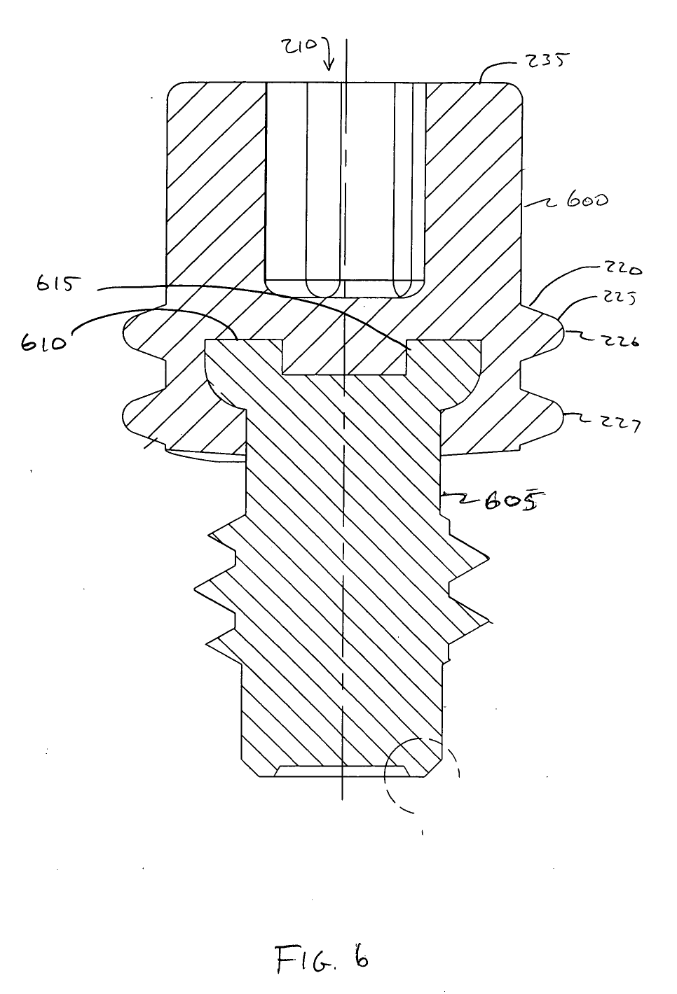

Referring now to the cross-section shown in FIG. 6, in an example, the screw is formed from separate shaft and head components. In an example, an insulative head piece 600 is molded over a shaft member 605, which is optionally conductive. In an example, the head 600 is insert-molded onto the shaft member 605. In another example, the head is assembled, adhered, press fit, heat staked, sonically welded, or otherwise coupled to the shaft. In an example, the head 600 is formed from Polyetheretherketone (PEEK), polycarbonate, or a ceramic. In an example, the shaft is formed from titanium or stainless steel. In an example, the shaft member 605 includes features 610, 615 that transmit torque from the head 600 to the shaft member 605.

FIG. 7 shows a perspective view of an example shaft member 605 that includes four protruding member 705, 710, 715, 720. In an example, the protruding members have surfaces 725, 730, 735, 740, 745, 750, 755, 760 that are approximately parallel with the shaft axis 701. The surfaces are arranged to transmit torque from an overmolded head to the shaft member. The surfaces define an opening which is filled with polymer when the shaft is molded into the head. In an example, the surfaces are approximately perpendicular to each other.

FIG. 8 shows a top-view of a medical device 801 including another example screw 800. In the illustrated example, the screw 800 is insertable into the back 802 of the device. In an example, the screw 800 includes a head 805 molded over a shaft 810. In an example, the head 805 is non-conductive. The shaft 810 is optionally conductive. In an example, the shaft 810 engages a connector 815 that includes an electrical conductor 812. In an example, the connector 815 includes an opening 820, into which the shaft is inserted to hold the connector in place in the device and in connection with an electrical contact 825 in the device.

In an example, the head 805 is insert-molded over the shaft 810. In an example, the head includes a shoulder 830 which engages an opposed surface 835 on a sleeve. The head 805 optionally includes one or more raised ribs 840 which press against the sleeve to form a seal that prevents body fluid from flowing into contact with the conductor 812 or electrical contact 825.

Another example screw is shown in FIGS. 9 and 10. The screw 900 has at least one compressible rib 905 extending around a head portion 910 of the screw. The illustrated example has three compressible ribs extending around the head 910. In an example, a shaft 920 is insert-molded or otherwise coupled to the head. In an example, the head has a cylindrical outer surface 925. In an example, the screw includes a cross-shaped drive interface 930.

Referring now to FIG. 10, the screw 900 is shown in an opening 1005 in a medical device casing 1010. In an example, the screw 900 includes a groove 1015 and an O-ring 1020 in the groove. The O-ring 1020 forms the compressible rib 905 (FIG. 9). A shaft portion 1025 of the screw engages threads in the casing 1010. An end portion 1035 of the shaft engages a conductor 1030.

FIG. 11 shows a cross-section of another example screw and sleeve system 1100. A sleeve 1105 includes an inwardly-extending rib 1115 that contacts an outer surface 1120 of a screw 1110. The rib 1115 creates a seal between the sleeve 1105 and the screw 1110. The sleeve includes an opening 1125 through which a wrench can be inserted to engage a drive interface 1130 in the screw.

FIG. 12 shows a cross-section of another example screw and sleeve system. A screw 1205 and sleeve 1210 are sized so that there is an interference fit between an outer surface 1215 of the screw and an inner surface 1220 of the sleeve. When the sleeve 1210 and screw 1205 are inserted in an opening in a device, such as an opening in a medical device casing, pressure between the sleeve and screw creates a seal between the screw and the sleeve, and between the sleeve and an inner surface of the opening in the device.

FIG. 13 shows another example screw. The screw 1300 includes an external drive interface 1305 and compressible ribs 1310 that press against in inner surface 1315 of an opening in a medical device casing 1320 or other component or device to create a seal between the screw and the device. In an example, the external drive allows for application of additional torque compared to internal-drive screws of similar size. The external drive also facilitates location of the screw and engagement of a wrench 1325 on the screw.

FIG. 14 shows another example screw 1400 assembled with a sleeve 1405 in a medical device casing 1410. The screw 1400 includes an external drive interface 1415 and a rib 1420 that presses against the sleeve.

FIG. 15 is a flow chart that illustrates a method. At 1505, a screw is inserted into an opening of an implantable device casing or other device. At 1510, a compressible member is inserted into the opening. In an example, a sleeve is inserted into the opening with the screw, and the sleeve is the compressible member. In another example, the screw includes a compressible member such as a compressible O-ring situated in a groove. At 1515, the compressible member is pressed against an inner surface of the opening with the screw. In an example, pressing the compressible member (the sleeve or O-ring, for example) creates and/or maintains a seal between the screw, the compressible member, and an inner surface of the opening. Optionally, at 1520, the screw is engaged with a lip on the sleeve. The lip prevents the screw from slipping out of the opening if the screw is not engaged with threads. In another example, friction between the screw, the compressible member, and the inner surface of the opening in the device holds the screw in place. At 1525, the screw is engaged on threads in the opening in the implantable device. In an example, the screw is advanced on the threads to engage a conductor and connect the conductor the device. At 1530, engagement of the screw is verified by tactilely detecting whether the screw is protruding from the opening. In an example, an operator such as a surgeon feels the screw with a finger to verify that it is engaged. In an example, the screw sticks out slightly when the screw is fully engaged, and the operator can confirm that the screw is in place by feeling for the screw. In another example, the screw is flush when fully engaged, and the operator can confirm that the screw is fully engaged by feeling for the screw and confirming that it is not protruding from the device.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A medical device comprising:
an implantable medical device casing including an opening having an inner surface;
a screw including a top portion including a driver interface, and a threaded bottom portion, at least one of the top portion and the bottom portion being non-conductive; and
a non-conductive sleeve configured to fit within the opening, the non-conductive sleeve having an outer surface and an inner surface,
wherein the sleeve and implantable medical device casing are adapted to sealingly engage with the screw when assembled.

2. The medical device of claim 1, wherein the screw comprises an insulative head that includes the top portion of the screw.

3. The medical device of claim 2, wherein the bottom threaded portion of the screw is conductive.

4. The medical device of claim 3, wherein the insulative head is insert-molded, assembled, adhered, press fit, heat staked, or sonically welded to the threaded bottom portion of the screw.

5. The medical device of claim 3, further comprising a conductor, wherein the screw is a set screw configured to couple the conductor to the implantable medical device.

6. The medical device of claim 1, wherein screw has an outer surface and at least one raised rib extending around the outer surface, the raised rib sealingly engaged with the sleeve.

7. The medical device of claim I, wherein the driver interface of the screw is an external drive interface.

8. The medical device of claim 1, wherein the non-conductive sleeve includes an inwardly-extending raised rib engaged against an outer surface of the screw.

9. A medical device comprising:
an implantable medical device casing having portions defining an opening;
a non-conductive sleeve in the opening, the sleeve having an inner surface defining an inner dimension; and
a screw at least partially contained within the sleeve, the set screw including an outer surface having a wide portion having an outer dimension larger than the inner dimension of the sleeve, and a narrow portion having an outer dimension smaller than the inner dimension of the sleeve, the wide portion sealably engagably with the non-conductive sleeve.

10. The medical device of claim 9, wherein the set screw includes at least one raised rib, the at least one raised rib including the at least one portion having an outer dimension larger than the inner dimension of the sleeve.

11. The medical device of claim 10, wherein the set screw includes a rib that extends around the screw to define a closed loop.

12. The medical device of claim 9, wherein the set screw comprises a threaded shaft and a non-conductive head connected to the threaded shaft, the non-conductive head including a raised rib protruding radially outwardly from the non-conductive head.

13. The medical device of claim 9, wherein the screw is engageable and disengageable with the implantable device, and the screw is retainable in the opening by the sleeve when the screw is disengaged from the threads.

14. The medical device of claim 13, wherein the sleeve includes a lip that extends inwardly over the screw and is engageable against the shoulder.

15. The medical device of claim 9, wherein the inner surface of the sleeve is generally cylindrical and defines an inner diameter, the wide portion of the screw having an outer diameter that is larger than the inner diameter of the inner surface of the sleeve.

16. The medical device of claim 9, wherein head includes an opening defining an internal drive interface.

17. The medical device of claim 16, wherein the sleeve includes a first end contained within the implantable device and a second end including an orifice through which the driver interface on the screw is visible.

18. A medical device comprising:
an implantable device casing including an opening having an inner surface; and
a screw having a threaded shaft and a non-conductive head coupled to the threaded shaft, the non-conductive head having an outer surface and at least one compressible raised rib extending around the outer surface;
wherein the screw is adapted to sealingly engage with the implantable medical device casing when assembled.

19. The medical device of claim 18, wherein the screw includes a groove in the outer surface and an O-ring situated in the groove, the O-ring including the compressible raised rib.

20. The medical device of claim 18, wherein the non-conductive head is insert-molded over the threaded shaft.

21. The medical device of claim 21, wherein the outer surface of the non-conductive head is cylindrical.

22. The medical device of claim 18, wherein the non-conductive head includes an external drive.

23. The medical device of claim 18, wherein the screw is engageable against a connector that is insertable in the medical device casing, the non-conductive head protruding out of the opening in the medical device casing when the screw is engaged against the conductor.

24. A method comprising:
inserting a screw having a non-conductive head into an opening in an implantable device casing;
inserting a compressible component into the opening; and
pressing the compressible component against an inner surface of the opening, wherein the screw and compressible member sealingly engages the implantable device casing.

25. The method of claim 24, wherein inserting a compressible component into the opening includes assembling an O-ring into a groove the non-conductive head of the screw, and inserting the O-ring into the opening with the screw.

26. The method of claim 24, wherein inserting a compressible component into the opening includes inserting a compressible sleeve into the opening.

27. The method of claim 26, further comprising engaging the screw with a lip on the sleeve that prevents the screw from moving out of the opening.

28. The method of claim 24, further comprising engaging the screw on threads in the opening in the implantable device.

29. The method of claim 24, further comprising verifying engagement of the screw by tactilely detecting that the screw is not protruding from the opening.
